(19) United States
(12) Patent Application Publication
(10) Pub. No.: US 2019/0272915 A1
(43) Pub. Date: Sep. 5, 2019

(54) System and methods for alternate-path access to medicare advance care planning education and conversation benefits on-demand by non-patients within home and other non-medical community settings

(71) Applicant: Ivan Keith Tolbert, Richmond, VA (US)

(72) Inventor: Ivan Keith Tolbert, Richmond, VA (US)

(21) Appl. No.: 16/501,090

(22) Filed: Feb. 21, 2019

Related U.S. Application Data
(60) Provisional application No. 62/710,625, filed on Feb. 22, 2018.

(51) Int. Cl. G16H 40/20; G16H 10/60

(52) U.S. Cl. CPC G16H 40/20; G16H 10/60

(57) ABSTRACT

The present invention relates to systems and methods for creating an alternate pathway to access, via a Stand-Alone Health Provider Entity, federally-compliant Medicare Advance Care Planning education and conversation benefits off-site of a doctor's office, hospital, or other medical facility. Access to this Medicare benefit is generally not available in an outpatient medical environment where most physicians don't have the time required to competently conduct in-depth one-on-one medical planning conversations with treated individuals, non-sick patients and family members, and where attorneys usually don't possess the requisite knowledge or skills to do so. The present invention enables, and herein discloses, a novel alternate path that provides more ready access to the existing Medicare Advance Care Planning education and conversation benefit and, thereby, substantially increases the potential number of eligible Americans who can competently complete professionally-guided medical planning conversations and legally create personalized and medically-useful advance directives and physician-authorized medical orders.

### CROSS-REFERENCE TO RELATED APPLICATIONS

This application claims the benefit of Provisional Patent Application Ser. No. 62/710,625 filed 2018 Feb. 22.

### BACKGROUND OF THE INVENTION

### 1. Field of the Invention

It has been a longstanding goal of the U.S. government and the medical community generally to increase the overall level of advance care planning participation among American adults, with formal government impetus toward that end legally codified three decades ago upon enactment of the Patient Self-Determination Act of 1991. From the beginning this has been a daunting challenge and an elusive goal, with the estimated number of Americans who had completed advance directives back then staying unchanged for the past 30 years, and still remaining today at only about 26%. Since that time, the increasing complexity of medical technologies, greatly expanded treatment options, available artificial life support choices that did not even exist 40 years ago, and the nexus of informed patient consent within the context of changing state laws, the ability of the average citizen to navigate this complexity, understand these options, surmount the information overload barrier, and make informed medical treatment and planning decisions has become even more difficult over time. Most people simply do not possess the breadth of information, or the ambient knowledge, required to competently engage these complex discussions and have advance care planning conversations either with their family members or with their doctors. Neither are they psychologically prepared to do so. In the U.S., members of society live in what practitioners with ordinary skills in the art describe as a “death avoidance culture,” wherein most citizens reflexively avoid difficult, and often painful, end-of-life discussions. It is this essential characteristic of the ACP conversational process that the present invention aligns particularly with U.S. Patent Class 434/237. The national need for higher-quality and more practically useful ACP health education strategies has become increasingly urgent. Increasingly, therefore, these conversations must be facilitated by knowledgeable intermediaries who can parse the information and psychologically guide the sensitive medical planning conversations necessary to ultimately achieve the goal: person-centered informed consent concerning future care decisions. Before ever getting sick, being admitted to a hospital, or awaiting an appointment with his or her regular primary care physician, using the present invention any Medicare-covered individual and her or his family member(s) can schedule, on-demand, a comprehensive, medically supervised, and professionally-competent medical planning conversation, deliverable at a convenient time and comfortable location independent of any medical facility, physician's office, or existing doctor-patient relationship. The stakes for not discussing, making, and documenting these critical future medical decisions well in advance has never been higher, nor have the individual risks involved in not doing so ever been greater. In less than a single generation, modern medical technology has so quickly progressed, that it has fundamentally changed the reality of serious illness, death and dying in America: Whereas it was universally true 50 years ago that a sudden cardiac arrest, breathing cessation, or an inability to swallow would have led inevitably to a quick death, today none of this is any longer true. Artificial Life Support technology has changed that, and in the process brought forth the urgent need for all American adults, and indeed for all others in advanced societies throughout the world, to anticipate the possibility of losing communication/decisional capacity and being unexpectedly “suspended” somewhere in a netherworld, stuck indefinitely between life and death in an undesirable state where they will never again “live” a quality life, but who also are not legally permitted to die a natural death. Most Americans who do not have an executed advance medical directive or an appointed Medical Power-of-Attorney, are unaware of this tectonic shift in the healthcare landscape and do not understand that they may be only one serious accident or illness away from entering this suspended life-and-death state themselves. At the time of this writing, thousands of Americans who did not have advance care planning conversations or create the necessary legal documentation are being kept alive only by means of artificial life supports in medical facilities throughout the nation, in most cases without their own consent or the consent of their families, sometimes for many years with no hope at all of ever leaving their permanent hospital bed. Had they ever been asked, few, if any, of them would have deliberately chosen such a fate for themselves. There is another serious challenge posed by this dilemma: Cost. Medical treatment and care costs for individuals in this state is tremendously expensive. In one world-famous case that eventually was adjudicated by the U.S. Supreme Court, the overall cost of care for a patient who spent 15 years incapacitated and on life supports totaled approximately $45 million, most of which was borne by U.S. taxpayers. In today's dollars, this cost would approximate $75, million.

At the time of this writing, more than 60 million Americans were enrolled in the federal Medicare program. With an estimated 10,000 more Americans reaching 65 years of age every day, this enrollment number is expected to swell to 72 million by 2025. It has been estimated that 75% of all healthcare expenditures in the U.S. are spent on 25% of the population at or near the end of life. Nearly half of all patients in the U.S. lose capacity and the ability to communicate for themselves at some point before they die. Even among the 26% of Americans who have completed an advance medical directive, the vast majority of them have not had a in-depth medical planning conversation either before or after executing the legal document. Medical professionals often refer to advance directives created absent a medical planning conversation as being “useless,” since the appointed Medical Power of Attorney usually has no idea whatsoever about what the patient would or would not want to be done regarding medical procedures and the treatment option decisions that must be made and must, therefore, resort to “guesswork.” In fact, most of these less-than-useful-when-the time-comes legal documents were created by attorneys as part of a generic estate planning process without the input of, and outside the presence or purview of, medical professionals. Consequently, they typically are devoid of comprehensive medical planning conversations of any kind concerning comparative medical options, treatment procedures, available choices, or the likely efficacy or not of potential outcomes from the medical decisions that will one day fall to the advance medical directive's owner or to the individual that has been appointed thereby as her or his Durable Power of Attorney for health care decision-making, and that make such legal documents “useful.” Therefore, it is important to draw a clear distinction between the advance medical directive legal document and the advance care planning education and conversational process that should underly it, and that is the content of the Medicare benefit, and that is the object of the presently disclosed invention. As stated in Redefining the “Planning” in Advance Care Planning: Preparing for End-of-Life Decision Making” in order to make clear this distinction, the authors write: “. . . the objective for advance care planning ought to be the preparation of patients and surrogates to participate with clinicians in making the best possible in-the-moment medical decisions . . . [and] for clinicians to help patients and surrogate decision makers achieve this objective in the outpatient setting. Preparation for in-the-moment decision making shifts the focus from having patients make premature decisions based on incomplete information [advance directive legal document] to preparing them and their surrogates [educationally and psychologically] for the types of decisions and conflicts they may encounter when they do have to make in-the-moment decisions [ACP education and conversation]. Advance directives, although important, are just one piece of information to be used at the time of decision making.” [emphasis added] (R. L. Sudore and T. R. Fried, Annals of Internal Medicine, 2010, American College of Physicians)

In light of these realities, this societal context, and the overarching importance of comprehensive education and medical planning conversations—with or without executing any legal document—both to improve treatment outcomes and to reduce the overall rapid rise in healthcare costs, the Centers for Medicare and Medicaid Services (CMS) created, in 2016, a new educational benefits category for all Medicare Part B beneficiaries nationwide: Professionally supervised Advance Care Planning conversations. Pointedly, this benefit is for the advance care planning conversation, and not the advance medical directive legal document. Sixty million Medicare beneficiaries instantly became eligible, and yet, because of resistant barriers inherent within American medical systems and practices nationwide, almost none of them are able to readily access and use their important new benefit.

According to a Cambia Health Foundation study (April, 2016), to date, only 14 percent of practicing physicians are cooperating in the delivery of this benefit to which their Medicare patients are legally entitled (and very few are motivated or properly trained to deliver this benefit, in any case). Consequently, primary care physicians constitute an often impenetrable barrier to their patients' own legal right (under the U.S. Patient Self-Determination Act of 1991) to access this federally guaranteed benefit, which by law must be made freely available by states to all adults within a citizen's own discretion only, and immediately upon the patient's sole volition, initiation and individual demand. Most states developed obtuse and densely worded legal directive documents and posted them on rather obscure websites in order to at least minimally comply with this federal legal mandate.

The undisputed best-practice model leaders throughout the U.S., Australia, Singapore and several European nations is the Respecting Choices® Advance Care Planning (RC) professional training and certification protocol. (https://respectingchoices.org) Of the three training/certification levels available within RC (First Steps, Next Steps and Advanced Steps), only the Advanced Steps level of training and certification is medically, professionally and ethically indicated and appropriate for the Medicare population of Americans aged 65 or over, or those with a disability. Advanced Steps is based on another widely accepted best-practice protocol—The National POLST Paradigm—a medical orders regimen that is now operating (or being implemented) in 47 U.S. states. (polst,org). In these and all other best-practice advance care planning conversation model programs that are known to those with ordinary skills in the art, competent advance care planning conversations require more than a few hours of conversation with patients and their families, and usually require multiple meetings to adequately complete. Still, the average conversation length among the scant 14% of physicians who have actually billed Medicare under this new advance care planning benefit is of an implausible 30 minutes duration (see below).

The systems and methods of present invention resulted from two years of research and development effort initiated shortly after establishment of the Medicare ACP education and conversation benefit in 2016, when it was discovered that a Part B Medicare beneficiary was unable to access this new benefit following a request for same made to his primary care physician. Instead of a qualified ACP conversation under his existing Medicare coverage, he was given an informational pamphlet and a standard boilerplate advance medical directive form to complete on his own. He also was unable to obtain this benefit from a local hospital with trained ACP facilitators, owing to the fact that he was not an admitted patient. Locked out of the benefit due to the lack of a means to access it, he appealed to another independent ACP facilitator who provided him the service but was unable to submit an insurance claim to Medicare because he was not already a Medicare-enrolled provider and was unable to comply with stringent regulatory mandates to qualify a claim under the new Medicare ACP conversation benefit's regimen. It stood to reason, then, that if this gentleman could not access the Medicare ACP benefit to which he was entitled because his own doctor had neither the time nor the inclination to perform it, then he was not alone in this dilemma and likely hundreds of thousands, if not millions, of similarly situated Medicare Part B beneficiaries were already confronting, or surely will confront, this same barrier to benefit access. It instantly appeared as a problem begging an invented solution. As far as it has been able to be objectively determined there now exists no other geographic area anywhere in the nation, other than where the systems and methods of the present invention were researched, developed, tested and re-tested, where an eligible Medicare Part B beneficiary may directly access this Medicare-qualified coverage benefit locally and from outside of his or her own doctor's office, or (possibly) an outpatient medical facility, since under Medicare regulations the benefit must be supervised by an actively licensed medical practitioner.

There is an immediate and urgent societal need to overcome these severe access barriers and practical impediments if the nation's goals of increasing participation in comprehensive medical planning conversations, ACP education, and the creation of competent advance directives and medical orders is ever to be realistically achieved. Instant disclosure of the methods and systems pertaining to the present invention that can effectively obviate these access barriers by creating a dedicated Stand-Alone Health Provider Entity and alternate access pathway specifically invented for this purpose, and thereby increase the overall number of Americans who may attain and use this valuable benefit to express and document their future medical preferences and decisions, will make an immediate and lasting contribution to improvements in the existing state of the current art, while also improving person-centered informed consent, patient treatment outcomes, and achieving desirable reductions in the rapidly escalating costs within the American healthcare delivery system overall.

### 2. Description of the Related Art

It is well understood among those with ordinary skill in the art that conducting advance care planning services with non-sick individuals, patients, caregivers, and family members can offer a powerful, effective, and beneficial means for improving health treatment outcomes, bolstering person-centered truly informed medical consent, reducing the rising costs of health care (especially at the end of life) overall by mitigating against the use of unwanted (and in too many cases futile medical treatments and procedures that are not desired either by patients or their caregiving family members), early empowering individuals regarding their future medical treatments in the case of incapacity, and significantly improving the frequency and quality of doctor-patient communications. Despite these far-reaching and well-known benefits, the expansion of professional advance care planning has been a tremendously daunting challenge within the U.S. health system for many years. While there are a number of contributing factors to the slow uptake rate for ACP services, chief among them are the structural access barriers that discourage ready delivery of these services by medical professionals and institutions: lack of available time among physicians to conduct one-to-one in-depth conversations with their patients, fee-for-service payment rules, financial disincentives confronting hospitals and medical systems, and a lack of awareness and/or interest by a huge swath of the general public at large. And yet, there remains only one available pathway for individuals to access their federal and state legal right to engage in these ACP services and create a responsive advance medical directive as a result: Their primary care physician and/or hospital admission. Others have made contributions to increasing the societal goal of improving overall health, wellness, and prevention progress through a number of innovations. Among them are Angelides's U.S. Pat. No. 10,199,126, Systems and Methods for Developing Individualized Health Improvement Plans. While targeting individuals with chronic health conditions as many ACP services also do, the computer-based health improvement technology presented in Angelides does not address future medical decision-making nor involve face-to-face patient meetings or discussions (even via telemedicine delivery systems), a standard attribute of ACP services that is even legally mandated under the federal Medicare ACP education and conversation benefit regulations. The presentation by Summerell, Rom, Roach, et. al., of a System and Method for Developing and Selecting a Customized Wellness Plan, U.S. Pat. No. 5,937,387, also makes an important contribution to the art by allowing for individual customization of a wellness plan, much as the present invention does with its own focus on individual ACP conversations, customized advance directives, and medical care plans. But, like Angelides, Summerell also is a computer-based technology system embuing the very same limitations vis-à-vis advance care planning as does Angelides—no individual face-to-face patient engagement and no impact on future medical decision making. The present invention employs a standardized conversation script tool for ACP consistency, but that also is designed to ease patient fears, lesson anxiety over what is sure to be a difficult discussion about medical interventions and end of life scenarios, and to deliver a measure of psychological comfort by engaging the conversation in “happy thoughts,” much as is the aim in Harris's Personal Communications Tool and Method of Use, U.S. Pat. No. 6,830,454, for encouraging therapeutic relaxation. The Harris tool, however, is a physical device apparatus that has no related bearing in any way on future medical decision making, care plans, or advance care planning services. Prior efforts at creating community-based, non-medical systems that can consistently provide high-quality ACP education and conversation services have met with spotty and only limited success. A study of one such effort, Implementing Advance Care Planning: A Qualitative Study of Community Nurses' Views and Experiences, cited poor teamwork, difficulty in aligning patient and family member views, difficulties in talking about death and dying, and a dearth of practical advice about ACP communication and documentation as shortcomings that are particularly challenging (Seymour, Almack, and Kennedy, BMC Palliative Care, 8 Apr. 2010). Another study examining community-based ACP efforts, Barriers and Facilitators for General Practitioners to Engage in Advance Care Planning: A Systematic Review, noted such barriers as the lack of physician skills to deal with patients' vague requests, difficulties with defining the right moment for ACP conversations, a prevalent attitude that it is the patient's responsibility to initiate the ACP conversation, and physicians' general fear of dashing patients' hope (Vleminick, et. al., Scandanavian Journal of Primary Health Care, Vol. 13, 2013, pp. 215-226). Two key observations of the study Anticipating Emotion: A Qualitative Study of Advance Care Planning in the Community Setting were 1) “. . . discussions about the end of life are sensitive and often difficult for health professionals, particularly those in the community setting” (even as it also is widely argued by those with ordinary skill in the art that it is community-based primary care physicians who should be the preeminent owners of the ACP conversational process), and 2) “The findings clearly depict two of the challenges faced by GPs and DNs in the community; emotional labour, and balancing patients' and families' expectations about care provision in the community with the limited resources available” (Minto and Strickland, International Journal of Palliative Nursing, Vol. 17, No. 6, 28 Sep. 2013) both argue the need for the kind of psychological/emotional support and sensitivity required for ACP conversations as represented by the present invention. There also have been innovative trials of community-based ACP conversations, in a whole-community-action-mobilization campaign (Waller, et. al, Increasing Advance Care Planning: The Need for Action at the Community Level, BMC Public Health, 9 May 2018) and in a two-day workshop format (Rabow, et. al., Advance Care Planning in Community: An Evaluation of a Pilot 2-Session, Nurse-Led Workshop, American Journal of Hospice and Palliative Care, Vol. 36, No. 2, 28 Aug. 2018.) both of which were judged to be less-than-successful and neither of which approximates the innovative approach, scope or principles of the present invention. The object of the present invention is to create a Stand-Alone Health Provider Entity and alternate access to professional advance care planning education, conversation, and psychological/medical support services via systems and methods of use that invent a new pathway that provides a useful result by effectively navigating around, over, and through existing medical-system-reality barriers that are not likely to be removed anytime in the foreseeable future. It will be appreciated by those with ordinary skill in the art that more such pathways to professional advance care planning education and conversation services like the one that is the subject of the present invention are desperately needed, and the sooner these alternate access pathways arrive, the better it will be for the public generally, the U.S. health care system particularly, and for all others who are concerned.

### SUMMARY OF THE INVENTION

The pressing matter addressed by the present invention's systems and methods (that will be appreciated first by those with ordinary skills in the art as being useful, novel, and unique, but also will be understood by them to be non-obvious in the construct and immediacy of its solution) is this widespread lack of immediate access to this new education and conversation benefit among the vast majority of eligible beneficiaries who need it and/or want it within the United States and its territories because most of them are not currently admitted under direct and present medical care in a hospital in-patient setting, an outpatient medical office, or an outpatient clinic where they might more easily seek to access it independently via their own self-initiative and timing were it not for intransigent barriers inherent in the American medical systems environment.

It is the contention of the present invention, as will be appreciated and understood by those with ordinary skills in the art as unique, novel, useful that this advance care planning conversation decision should be the patient's personal choice only, and should not be rendered dependent upon any physician's unilateral discretion. Nor does the present invention contend that this benefit should be subject to any physician's formal or informal veto power. The first embodiment of the present invention's systems and methods set forth what appears to be the one and only means for providing direct access to this medical education and conversational benefit without the need for direct participation by the patient's regular Primary Care Physician/Specialist, or admission to any medical facility, while also meeting the Medicare requirement for clinical supervision, but provided from outside of such an already existing patient-doctor relationship. By using the present invention's Stand-Alone Health Provider Entity for this single-purpose episode only, these inherent structural impediments may be overcome: All Medicare patients may now access the educational conversation benefit to which they are entitled, from right within their own homes, churches or other community locations without the rigid barrier effect of the patient's own regular non-participating primary care physician and without the need for admission first to any medical office, hospital or other inpatient medical facility. In the first embodiment, the present invention does not impact any doctor or health facility's present ability to deliver this education and conversation benefit directly to their already admitted/enrolled patients themselves (as indeed they should), but importantly the present invention neutralizes their barrier influence by creating an alternate method, systems, and means for Medicare-insured access to this rightful benefit even if these physicians and medical systems don't or won't participate. The systems and methods of the present invention are especially beneficial for historically underserved American patient population groups. As stated in Low Completion and Disparities in Advance Care Planning Activities Among Older Medicare Beneficiaries (Harrison, Adrion, Smith, et. al., Journal of the American Medical Association, Vol. 176, No. 12, December 2016): “While [Medicare] reimbursement is a critical step forward, effective, targeted approaches are needed to ensure increased completion of ACP among all older adults. Innovative ACP communication strategies are being developed both for minority populations and populations of older adults with multimorbidity and dementia. In the future, clinicians should use these tailored tools when discussing ACP with these particularly vulnerable groups.” The systems and methods of the present invention disclosed herein are among these innovative communication strategies that effectively create a Stand-Alone Health Provider Entity and provide an alternate path for more ready access to the Medicare ACP education and conversation benefit for these and other population groups that, heretofore, did not exist.

The dilemma addressed and ameliorated by the systems and methods of the present invention is more readily understood in view of these additional facts:
Medicare's regulatory requirements mandate that approved Advance Care Planning conversations must be medically supervised only by currently licensed health care professionals.
Demographically, the fastest growing population segment in the U.S. is the cohort aged 85 and older. Americans are living substantially longer, but they also are usually living with multiple chronic conditions, with the attendant strains on the healthcare system that such conditions entail. The older American population is expected to top 53 million by 2020 and to double by the year 2050.
There is no Place of Service requirement associated with this Medicare benefit.
The nation's predominant managed care fee-for-services profit model limits most physicians' face-to-face time with patients to approximately 15 minutes apiece, a woefully insufficient allotment of time to competently engage in any degree of minimally acceptable or professionally responsible medical planning conversation.
Within the extant fee-for-services profit model, the established Medicare reimbursement rates for Advance Care Planning Conversation benefits is generally considered too low to support clinical service time devoted by more highly compensated physicians and other health care practitioners. Therefore, very few of these qualified professionals and/or medical systems are trained, comfortable, motivated, willing or able to provide these comprehensive conversational benefits for their patients. The present invention's deployment of highly trained and certified advance care planning facilitators who work under the direct supervision of licensed medical practitioners at a lower compensation rate mitigates against this built-in financial disincentive.
All best-practice research into Advance Care Planning agrees that optimal values-based and person-centered medical conversations and decision making should occur well “upstream” in homes and other non-medical settings, long before the onset of any medical crisis, serious illness, or sudden injury when anxiety, mental depression, upset or worry can seriously confound clear thinking, learning, and decision making about future medical treatment options among both the patient and responsible family members, as well as their legal proxies.
Early evidence strongly suggests that medical systems are already “gaming the system” with regard to this new Medicare Advance Care Planning benefit, at the direct expense of U.S. taxpayers and patients.² Wasteful billing practices within the first two years of this benefit have resulted in nearly $200 million in charges for Advance Care Planning “conversations” averaging only 38 minutes (this according to Medicare's own published data), an impossibly short time period to engage in any substantive conversation or medical planning process. System “gaming” and waste of federal tax dollars also attains to the delivery of unqualified ACP conversations by individuals who are neither trained nor qualified to serve elder Medicare patients or those facing serious advanced illness, life-support decisions, and impending end-of-life, which befalls the vast majority of U.S. Medicare beneficiaries.
Tele-health delivery of Advance Care Planning services online was first permitted, on a limited basis, by the federal Centers for Medicare and Medicaid Services in 2018.
Medicare regulations prohibit hospitals and medical facilities from seeking reimbursement for delivering advance care planning education and conversational services to their inpatient populations, and hospitals generally do not allow “walk-ins” from the public of non-admitted individuals who may be seeking clinically supervised advance care planning services under their Medicare benefit.

It is another embodiment of the present invention, within the scope of its principles, to provide these educational, psychological, medical and conversational advance care planning services via use of computer-enabled technologies, including secured telemedicine hardware and software configurations, as well as websites and mobile device applications, to the extent permitted, and within the parameters established, by the federal Centers for Medicare and Medicaid Services.

It is another embodiment of the present invention, within the scope of its principles, to provide these educational, psychological, medical and conversational advance care planning services to Medicaid-insured individuals, to the extent permitted, and within the parameters established by each U.S. state. For example, California currently provides this benefit for both its Medicaid-enrolled and its Medicare-enrolled citizens.

It is another embodiment of the present invention, within the scope of its principles, to provide these educational, psychological, medical and conversational advance care planning services to ethnic minorities and other population subgroups using different languages and other cultural context cues.

It is another embodiment of the present invention, within the scope of its principles, to provide these educational, psychological, medical and conversational advance care planning services on site in the workplace of non-medical facilities and within a plurality of other common employment locations.

### BRIEF DESCRIPTION OF THE DRAWINGS

It will be appreciated by those of ordinary skill in the art that the drawings are for illustration only. The nature of the present invention, as well as other embodiments of the present invention, may be more clearly understood by reference to the following detailed description of the invention, to the appended claims, and to the several drawings.

FIG. 1 depicts a flowchart of one illustrative embodiment of receiving a request for advance care planning educational and conversational services by an individual, who may or may not have already been granted or denied services by his or her own Primary Care Physician, and the scheduling of a first appointment time and location with such individual requestor.

FIG. 2 depicts a flowchart of one illustrative embodiment of the present invention's processes associated with a first meeting for advance care planning education and conversational services, including disclosure of the present invention's proprietary tools and key medical planning decisions to be made by the requestor and recorded by the on-site facilitating healthcare professional.

FIG. 3 depicts a flowchart of one illustrative embodiment of the post-appointment activities of the facilitating healthcare professional using and transmitting the present invention's proprietary tools via computer-enabled means in a HIPAA-compliant (Health Insurance Portability and Accountability Act of 1996) encrypted internet portal environment, and computer based data server automated handling and routing of the received proprietary tools, for the purpose of acquiring clinician supervisor authorization and creating the requestor's customized advance medical directive.

FIG. 4 depicts a flowchart of one illustrative embodiment of the present invention's return path for a customized advance medical directive's delivery back to the on-site facilitating healthcare professional via computer-enabled means in a HIPAA-compliant (Health Insurance Portability and Accountability Act of 1996) encrypted internet portal environment, and the present invention's computer-enabled insurance claims processing steps for submission by an independent healthcare provider entity to the appropriate Medicare Administrative Contractor and/or to a plurality of other qualified insurers.

FIG. 5 depicts a flowchart of one illustrative embodiment of the present invention's process activities as undertaken by the facilitating healthcare professional during a follow-up second scheduled advance care planning educational and conversational meeting, wherein the requestors decisions, wishes and preferences are confirmed as accurately recorded and presented, as well as education about the customized advance medical directive's proper execution, use, and distribution is dispensed.

FIG. 6 depicts and discloses a graphical illustration and preferred in a first embodiment method of use for the Medicare ACP and POLST Paradigm Compliance Proprietary Tool 1, called the Acknowledgements Form, for ensuring signed client verification that legally mandated and regulatory disclosures and disclaimers were shared, discussed and understood before ACP services were initiated.

FIG. 7 depicts and discloses a graphical illustration and preferred in a first embodiment method of use for the Medicare ACP and POLST Paradigm Compliance Proprietary Tool 2, called the Quality versus Quantity of Life Preferences Scale, for determining an individual's deeply considered end-of-life preferences, CPR decision, ALS Trial Period, and Treatment Level choice.

FIG. 8 depicts and discloses a graphical illustration and preferred in a first embodiment method of use for the Medicare ACP and POLST Paradigm Compliance Proprietary Tool 3, called the Service Validation Voucher, for recording the essential medical treatment documentation, client evaluation and signature, supervising clinician evaluation and signature, conversation duration, and facilitator activity documentation and signature.

FIG. 9 depicts a cloud-level macro view of present invention's alternate access pathway, recommended for use as the front page drawing (without numbered or other cross references to the Detailed Description).

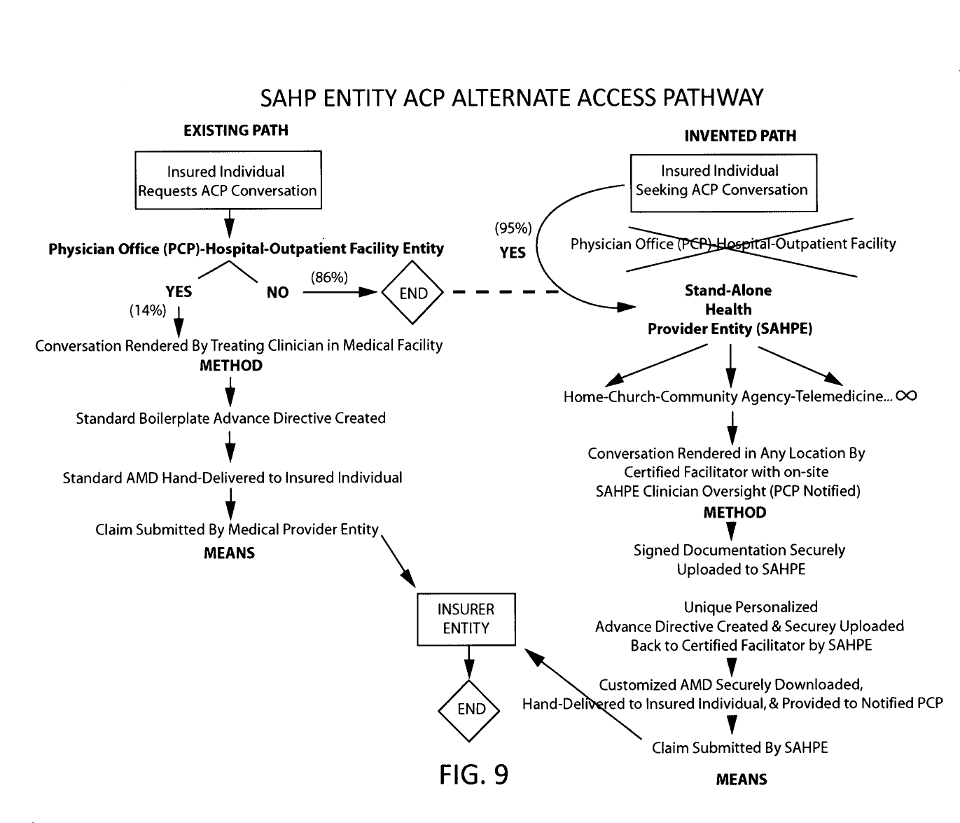

### DETAILED DESCRIPTION OF THE PREFERRED EMBODIMENT

### 1. Definitions

“Acknowledgements” means the official form for compliantly disclosing and disclaiming mandated legal information to clients and patients.

“ACP” means Advance Care Planning.

“ALS” means Artificial Life Supports.

“AMD” means Advance Medical Directive.

“Client” means, whether sick or well, Medicare-enrolled individual with Part B service coverage.

“CMS” means the federal Medicare program or its owner, the U.S. Centers for Medicare and Medicaid Services.

“CPR” means the standard U.S. medical procedure regimen for cardiopulmonary resuscitation in the event of sudden cessation of heartbeat and/or breathing.

“HCIP” means HIPAA-Compliant Internet Portal

“HIPAA” means the U.S. Health Insurance and Portability Act of 1996.

“MAC” means Medicare Administrative Contractor.

“MCS” means SAHPE-enrolled Medical Clinician Supervisor.

“QvQ” means Quality versus Quantity Life Preferences Scale.

“SAHPE” means Stand-Alone Health Provider Entity that is a Medicare-enrolled provider with approved access to the Centers for Medicare and Medicaid Services (CMS) Electronic Data Interchange systems (EDI) for eligibility verifications, claims processing, direct banking deposits, and other system-derived services.

“SCP” means SAHPE-enrolled Certified Professional.

“SW” means the proprietary Service Validation Voucher form used to capture the information elements for adequate regulatory reporting compliance of medical data and required documentation of qualified and performed medical services.

### 2. Discussion

The present invention discloses the systems, methods, business processes, tools, means, and essential knowledge required to successfully access, open and then navigate an alternate path to Medicare-qualified delivery of, and claims reimbursement for, an important and valuable new insurance benefit that in the current medical system environment, and heretofore, has been and remains practically inaccessible for the vast majority of eligible Medicare Part B beneficiaries, nationwide.

The method according to a preferred embodiment of the invention unfolds in six successive steps, described in consistency with the drawings:

### Step 1: Initial Beneficiary Contact

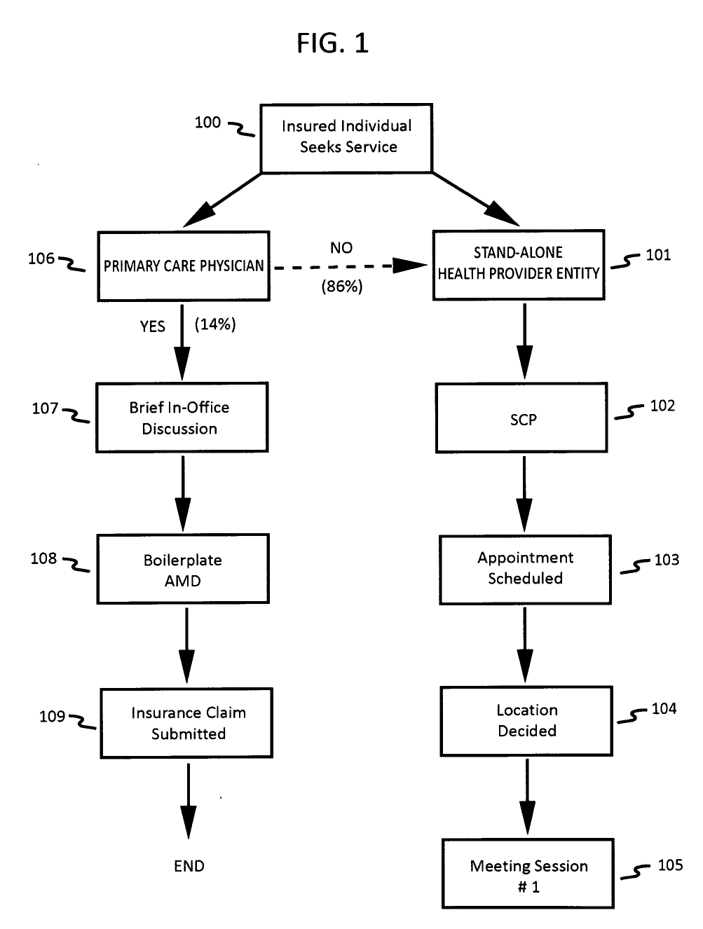

1.a. In the first step, and in accordance with FIG. 1, the system intakes a plurality of N number of Medicare Part B beneficiaries who are actively seeking to access their Medicare Part B advance care planning benefit, 100 or any one such beneficiary in a single instance of said intake who is seeking a plurality of available advance care planning solutions to learn, to make personal care decisions, and/or to appoint a surrogate decision maker in the event that he or she were ever to lose personal decisional capacity suddenly or gradually. Such seeking behavior may have emanated from a) personal felt need because of a recent medical or progressing diagnosis, b) recent observed medical experience or loved one's serious health decline/loss of life, c) recent first-time awareness of the need and legal right for an advance medical directive and advance care planning education and/or conversational services, d) denial or inadequacy of service response from the seeker's own Primary Care Physician or Medical Specialist 106, and/or any combination of the foregoing or other pluralities of personal motivating factor combinations.

In the alternative, whenever the seeker might get a favorable response from his or her own PCP 106, which occurs in only about 14% of the researched instances, then the client seeker will be subjected to a very brief discussion about advance directives 107, of an average 30.8 minutes in duration (2018 Medicare data), will be given a brochure about advance medical directives, may be given a boilerplate form to complete 108, or may be pointed to an Internet website where a boilerplate form may be downloaded and completed independently, the whole episode of which will be billed to Medicare 109, usually coincident with attached billing for another delivered medical service for which the client seeker originally visited the PCP in the first place.

1.b. In accordance with FIG. 1, the SAHPE 101 receives the service request and confirms that the seeker will be promptly contacted by a SAHPE-Certified Professional (SCP) 102 for service follow-up, to whom the potential client has been referred/assigned by the SAHPE 101.

### Step 2: Appointment Setting

2.a. In accordance with FIG. 1, the SCP contacts the potential client in order to schedule an acceptable appointment date and time 103, as well as to choose a mutually agreeable location 104, which may be the potential client's home, the SAHPE offices, a church, a public library, a public restaurant, a work site, or a plurality of other potential non-medical-facility locations.

### Step 3: Conduct of Conversation Meeting 1

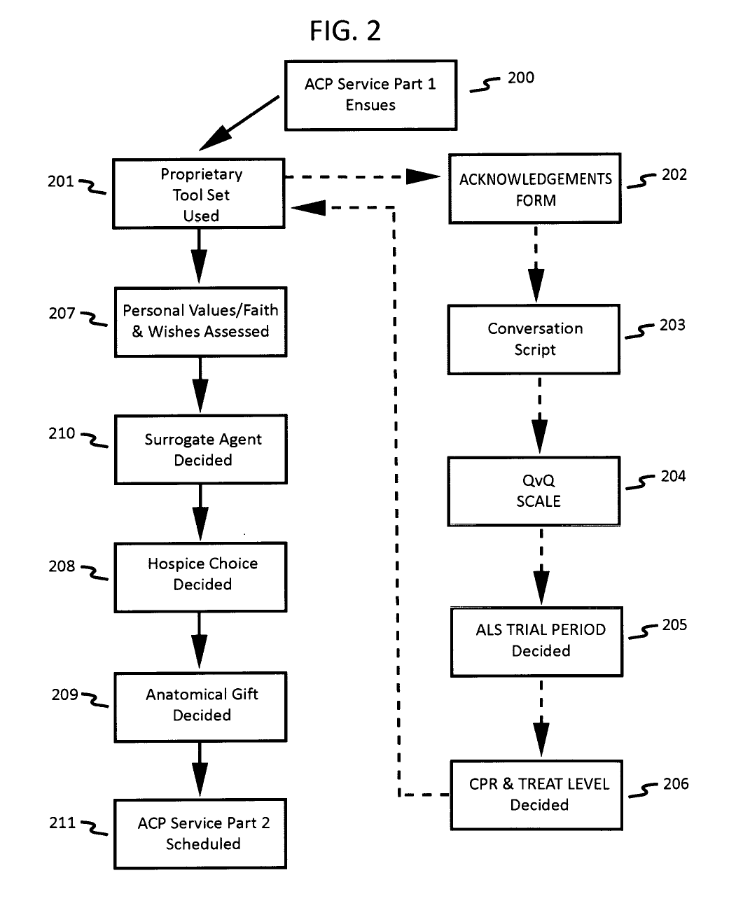

3.a. In accordance with FIG. 2, the SCP 102 professionally facilitates ACP conversation number one 200 with the client and any attending family member(s) or her or his invited participants by using the SAHPE's 101 proprietary tools and forms, including its Acknowledgements Form 202, Conversation Script 203, Quantity versus Quality of Life Preferences Scale 204, Artificial Life Support Trial Period Option 205, and CPR-Treatment Level Form 206.

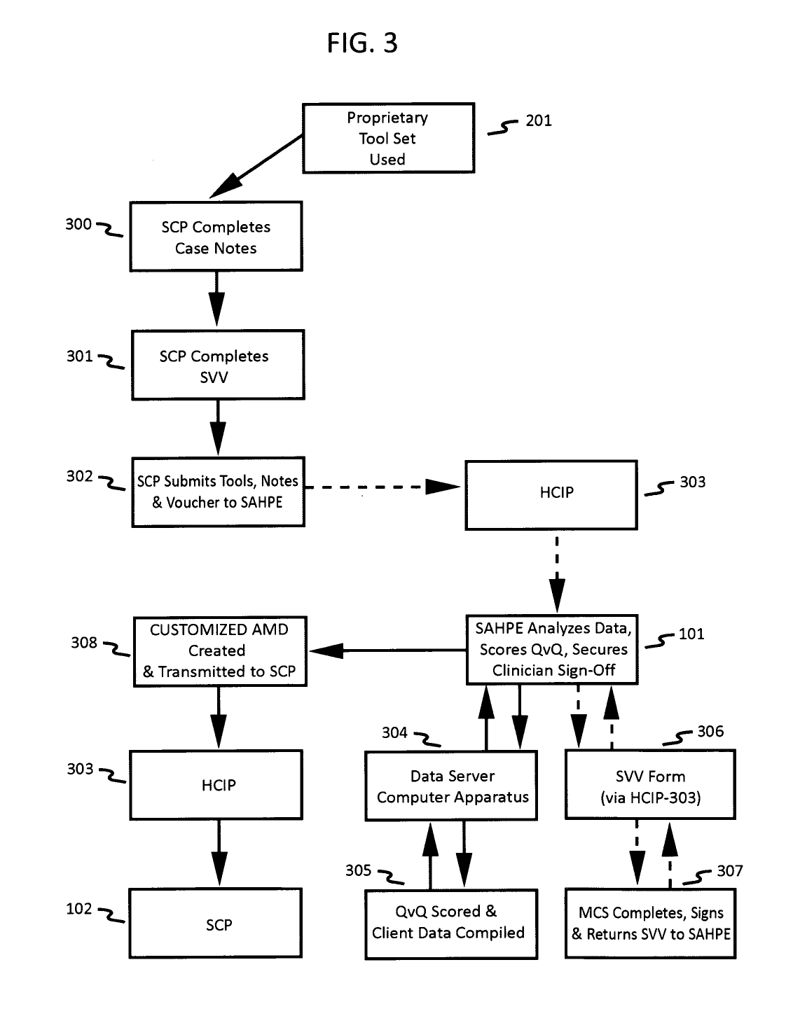

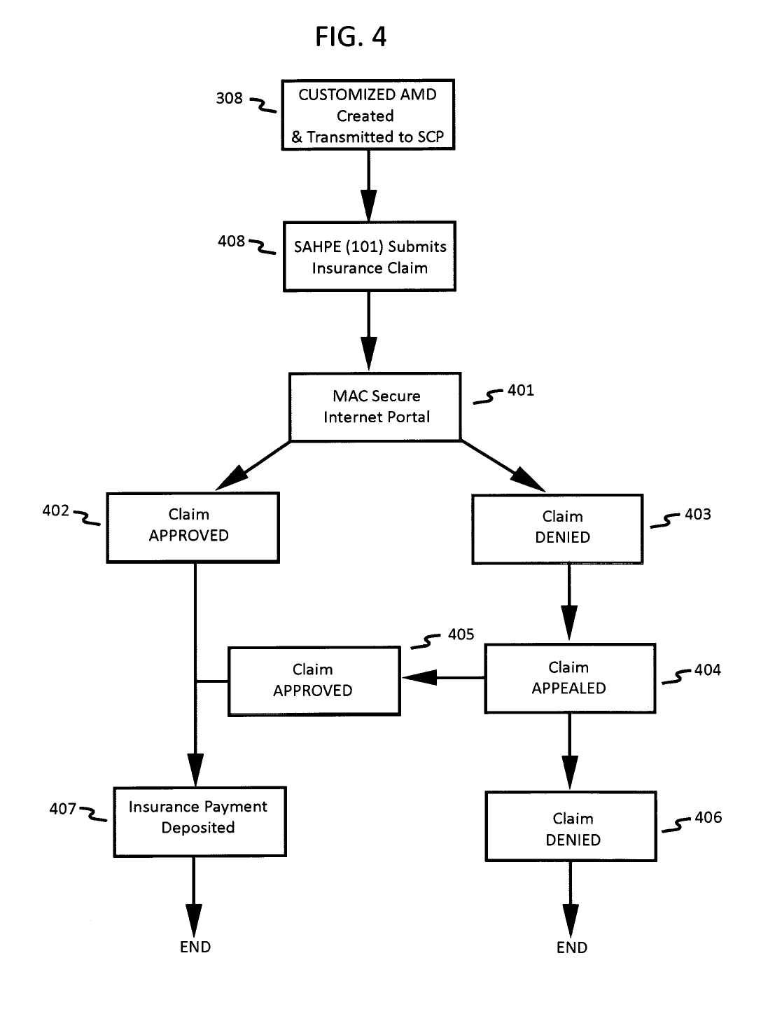

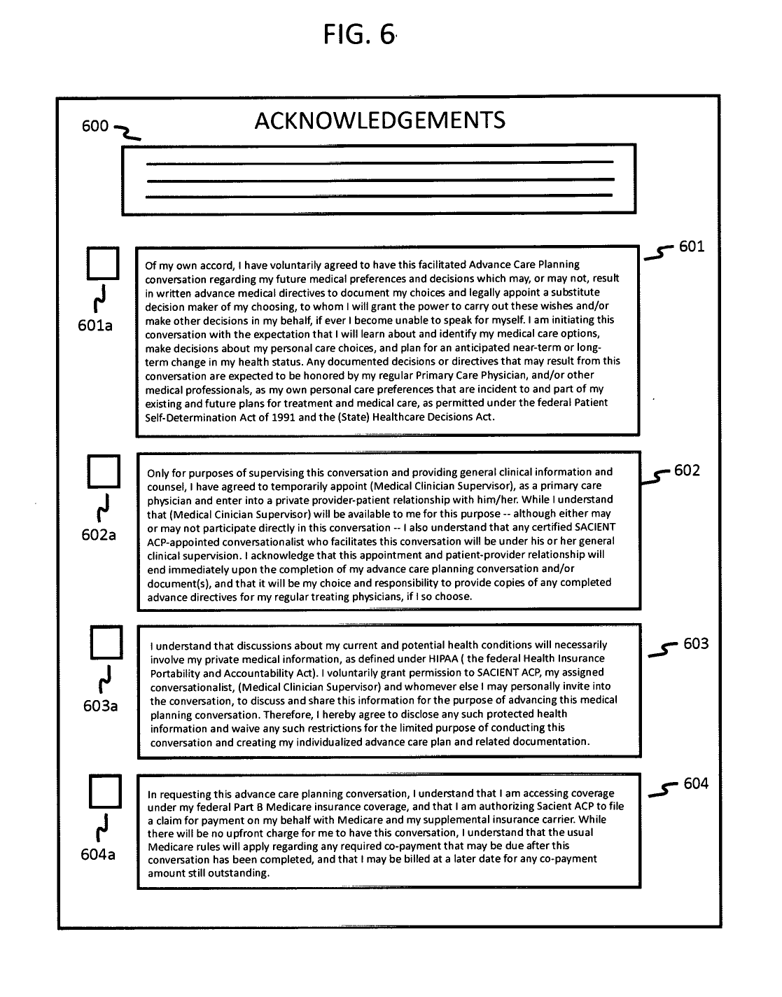

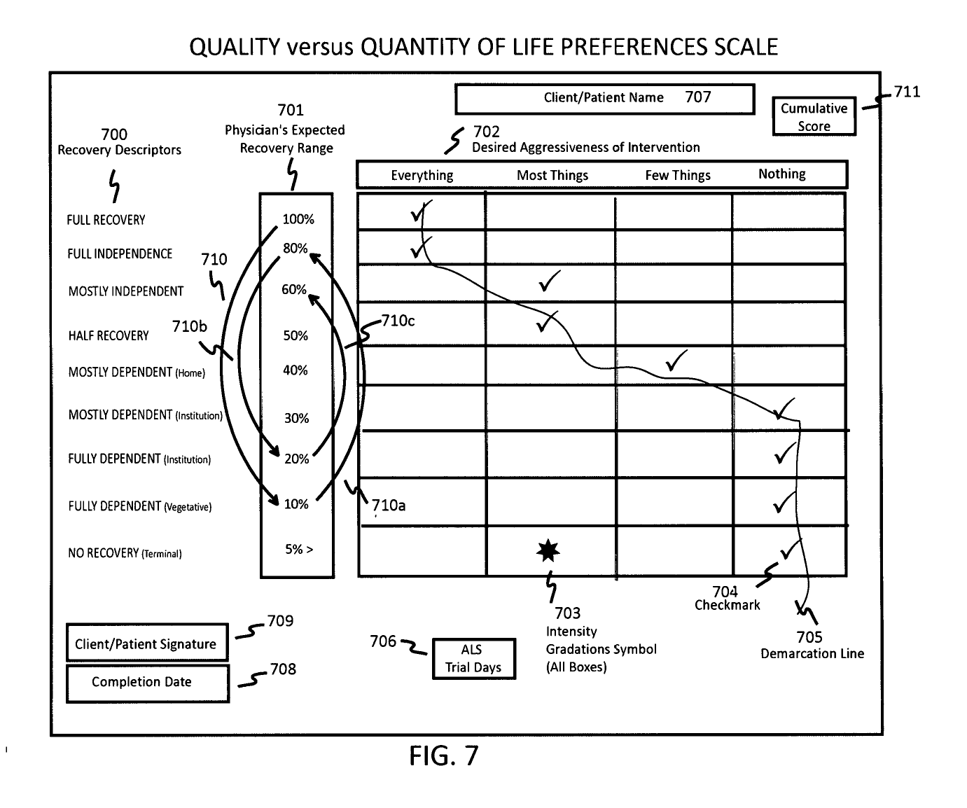

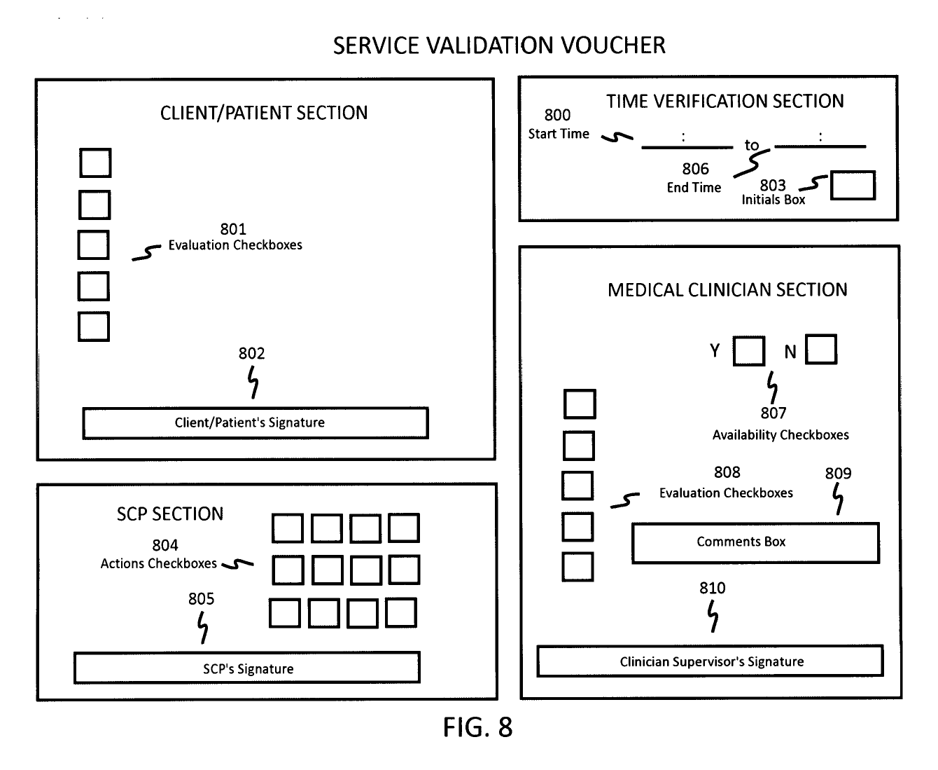

3.b. In accordance with FIG. 2, the SCP 102 first reviews, explains, and secures the client's signature on the Acknowledgements Form 202, before next using learned certification skills to educate, provide crucial information, and professionally guide the participants through the ACP conversation, while maintaining personal neutrality and also following conversation-one script prompts and triggers 203 about when to record notes that accurately document the client's personal values, faith preferences/world view, and special health care considerations 207, the CPR/Treatment Level decision 206, the hospice decision (indicating home or hospital preference) 208, the anatomical gift decision 209, appointment of a surrogate decision maker 210, in addition to the chosen trial period, if any 205, the QvQ results 204, and other items, e.g., preferred hospital choice.
3.b.1. In accordance with FIG. 6, once a potential client chooses to use the alternate access path of the present invention's systems and methods in order to obtain professional advance care planning education and conversational services, in the preferred embodiment a first proprietary tool, called the “Acknowledgements Form” is used in the following manner to set forth Medicare-mandated, and/or a plurality of other, legal understandings, disclosures and disclaimers, and to acquire the client's initialed and signed verifications that each said disclosure, disclaimer, and understanding was, in fact, set forth:
a. The client is encouraged to read the form instructions and its purpose 600, before the SCP 102 explains the content of the first paragraph 601, which informs that the ACP conversation process is “voluntary” per Medicare regulations, that the purpose is for considering future medical treatment options in anticipation of a near-term or a long-term change in health status, that completion of an advance directive legal form is not required and is not part of the Medicare ACP benefit, and it cites the federal and state statutory laws that guarantee the right of all American adults to direct their own future medical care in advance. The SCP 102 presents the document to the client if he or she wishes to read it, and is then instructed to check the box adjacent to the paragraph 601a.
b. Next, the SCP 102 explains the content of the second paragraph 602, which informs that Medicare requires that the ACP conversation process be clinically supervised by a licensed medical professional, and it sets forth the name of the medical professional who is providing clinical supervision and oversight of the present session. The SCP 102 further explains that this medical professional is immediately available to answer any general medical questions that may arise during the ACP conversation, that the client agrees to enter a single episodic patient-doctor relationship with this medical professional for the sole purpose of the ACP Conversation, that this relationship ends instantly and automatically upon conclusion of the ACP conversation process and service. The SCP 102 presents the document to the client if he or she wishes to read it, and is then instructed to check the box adjacent to the paragraph 602a.
c. Next, the SCP 102 explains the content of the third paragraph 603, which seeks the client's authorization and permission for the SCP 102, the supervising clinician, the SAHPE 101 and whomever the client invites into the ACP conversation to discuss and handle his or her Protected Health Information under provisions of HIPAA for this single episodic purpose only, and that this authorization ends instantly and automatically upon conclusion of the ACP conversation process and service. The SCP 102 presents the document to the client if he or she wishes to read it, and is then instructed to check the box adjacent to the paragraph 603a.
d. Next, the SCP 102 explains the content of the fourth paragraph 604, which explains that Medicare established the new ACP education and conversation benefit under the “normal co-pay” rules, and that the client, therefore, may be billed later for any outstanding co-pays or deductible amounts that may be due after the insurance claim has been submitted. The SCP 102 presents the document to the client if he or she wishes to read it, and is then instructed to check the box adjacent to the paragraph 604a.
   e. Next, the SCP 102 turns to the backside of the form and asks the client to sign and date it (not shown). The SCP 102 retains the signed form for later submission to the SAHPE 101.
3.b.2. In accordance with FIG. 7, once a potential client chooses to use the alternate access path of the present invention's systems and methods in order to obtain professional advance care planning education and conversation services, in the preferred embodiment a second proprietary tool, called the “Quality versus Quantity of Life Preferences Scale” 204 is used, for example, in the following manner to encourage client introspection into her or his most deeply held personal values for the purpose of attempting to answer this very difficult, but important, question about when “enough is enough” regarding aggressive medical treatments that threaten to lessen the client's overall quality of life: “When would life be no longer worth living . . . FOR YOU?” While not seeking to write “a hard line in the sand,” the QvQ Scale instrument 204 is intended to provide the framework for exploring this question that most people have not asked themselves before, but the answer to which is crucial for future medical personnel, family members, and appointed surrogates to know. It can only be properly answered by the individual him- or herself, and it lies at the very core of the whole advance care planning conversation process itself. The goal is to narrow the discussion to an approximate two-level range of quality-versus-quantity-of-life-outcomes, stratified along a continuum of 9 levels of possible recovery and 4 corresponding levels of desired (or not) aggressive medical interventions. The QvQ Scale instrument 204 also is used discuss and decide upon a possible trial period on life supports if the client chooses to establish one, or to decide, at the client's discretion, to leave the decision on the length any extended period of time on life supports to be made later by the client's appointed surrogate decision maker. The instrument's method of use is:
a. The SCP 102 engages the client in a hypothetical medical event wherein, for example, the client is told to image him- or herself having a normal day shopping at the mall when, suddenly, he or she collapses to the ground and is rushed unconscious to the nearest emergency department. The attending physician approaches family members and the client's appointed surrogate decision maker with the news that a cardiac arrest caused the medial crisis, that the client is sedated, that an emergency surgical procedure must be performed within the net 30 minutes to avoid a second event, and that the surrogate must decide and authorize the surgery.
b. While retaining the instrument 204 in his or her possession, displaying it to the client, and pointing to various sections of it while speaking, the SCP 102 tells the client that his or her surrogate will immediately pose two questions, for example, to the doctor: 1) How risky is the proposed surgical procedure and what are the odds for survival of it?, and 2) How much damage was done and what is the client's life going to be like after a successful surgery and medical discharge?
c. Next the SACP 102 points with a writing instrument or finger to the instrument's stratified-levels listing of expected recovery ranges 701, explaining that the doctor's answer to question one of b. above is, for example: “Not to worry! It's a routine minor surgical procedure we perform here every day. We'll make a small incision, clear out the blockage, stitch everything up, and in a month there'll hardly be a small scar visible. Your loved one will have no problem surviving the procedure.” Pointing to the ranges, the SACP 102 then tells the client that the doctor's answer to question two of b. above can vary among the ranges, with the doctor saying, for example, on the one hand: “Your loved one was very fortunate. As soon as she fell, a nearby person rushed and administered CPR, restoring the heartbeat in about a minute. I expect that your loved one will make a full 100% recovery and go back to living her life as if nothing ever happened.” Or, the doctor's answer, on the other hand might be quite the opposite, for example: “Unfortunately, your loved one was unconscious and without a heartbeat for a long time, nearly 40 minutes before the ambulance arrived. And because permanent brain damage begins to occur after only 5 minutes when this happens, sadly I suspect that the brain damage is severe. While your loved one will have no problem with the surgery, I expect that her life after this will never be the same. She won't be going home from here because I believe she will be in a persistent vegetative state and retain only about 10% of her former functionality. She will require around-the-clock medical and nursing care for the rest of her life, and she will not know who she is, where she is, or who is with her.”
d. Then the SCP 102 first points to the 100% recovery range and next to the continuum range of Desired Aggressiveness of Intervention 702, while, for example, asking “If you were expected to have a 100% full recovery and go back to your normal life, how much medical effort would you want the doctors to use in order to save your life . . . do everything, most things, a few things, or nothing at all?” When the client answers “everything,” then the SCP 102 places a checkmark 704 in the appropriate corresponding box space to record that choice in the 100% recovery range level row.
e. Next, the SCP 102 points near the bottom to the 10% recovery range level, where persistent vegetative state is briefly worded in the text description 700, and, for example, says: “Now what if the doctors told your loved ones that you would have a very poor recovery and have to spend the rest of your life in a nursing home . . . not knowing who you are, where you are, or who is with you . . . how much effort would you want the doctors to use in that case to try and save your life?” When the client answers “nothing,” then the SCP 102 places a checkmark in the appropriate corresponding box space to record that choice in the 10% recovery range level row. The SCP 102 continues to use this “see-sawing” technique 710-710c to walk the client successively through the remaining ranges, placing checkmarks appropriately along the way, until only two recovery range levels remain.
f. Next, for example, the SCP 102 points to the last two unfilled recovery ranges and explains that determining the client's broad range of quality-versus-quantity-of-life personal values outcomes has substantially been achieved, and that the remaining two rows reflect approximately the same health conditions, with the higher recovery range of the two (e.g., 50%) representing the ability to receive the same level of needed care while at home, but the lower recovery range (e.g., 40%) means the client will get the same amount of care, but in a nursing home. Then the SCP 102 asks the same Desired Aggressiveness question 702 and records the last two answers accordingly in order to complete all checkmarks 704. Then the SCP 102 draws a demarcation 705 line through and connecting the checkmarks to create a visual curve of the client's combined responses.
g. Next, the client is asked to consider a length of time, expressed as any number of days, the she or he may wish to remain in an incapacitated state on artificial life support systems, in order to establish a possible Trial Period. For example, the SCP 102 explains this as an option that is not required, and further explains that the power to choose the length of any extended period on life supports can be left to be decided if and when the time comes by the client's appointed surrogate decision maker. If the client chooses a trial period, the SCP 102 records the number of days in the QvQ Scale's 204 field box 706. If the client chooses to leave the decision to her or his surrogate decision maker, then the SCP 102 enters “Trial by Agent” in the same field box 706.
h. Next, the SCP 102, enters the client's name 707, the date 708, and requests the client's signature 709.
i. Later, following the conversation's end, the SCP 102 will calculate and enter a Cumulative Score for the instrument in the field box 711, weighting this score by a plurality of observed factors, each with pre-assigned values, including evident depression, force of expression (from ebullience to reluctance), degree of independent judgment, et. al.
3.b.3. In accordance with FIG. 8, once a potential client chooses to use the alternate access path of the present invention's systems and methods in order to obtain professional advance care planning education and conversation services, in the preferred embodiment a third proprietary tool, called the “Service Validation Voucher” 301 is used in the following manner to record proof of a face-to-face service delivery, to record appropriate medical documentation as may be required by Medicare, to record required signatures, to record the time duration of the ACP conversation, to evaluate the service delivery and performance of the SCP 102 from both the client and the clinician perspectives, and for the SAC 102 to record attendees, actions taken during the ACP conversation, and planned follow-up actions. The method of use is:
a. Upon conclusion of the ACP education and conversation session, the SCP 102 reads aloud to the client a series of evaluative questions listed on the SW, records the clients answers by marking the appropriate checkboxes 801 and then requests the client's signature on the appropriate line 802.
b. Next, the SCP 102 completes the facilitator form section by marking the appropriate checkboxes 804, and signs the form on the appropriate line 805, before completing the time duration fields 800, 806 and requesting that the client enter his or her initials in the appropriate field box 803.
c. By the method of FIG. 3, the SCP 102 submits 302 the SVV form to the SAHPE 101 for additional processing.
d. By the method of FIG. 3, the SAHPE 101 enters the SW form data into a computer-enabled digital storage data-server apparatus 304 for aggregation with the client's other received and stored data 305 which has been scored and logged, before using the method of secured transmission 303 to transmit the form copy 306 for review and signing by the supervising medical clinician 307, whereupon the same secure transmission method 303 is used to return the form to the SAHPE 101.
e. By the method of FIG. 4, upon receipt by the SAHPE 101, the now authorized SW form data is used as the basis for preparing and submitting an insurance claim 408, and processing the claim in accordance with FIG. 4 401-407.
f. The SW form data is combined with additional aggregated, in accordance with Step 4 below, client data in order for the SAHPE 101 to create and transmit a customized advance medical directive 308 to the SCP 102 by the secure method and means of FIG. 3 303.

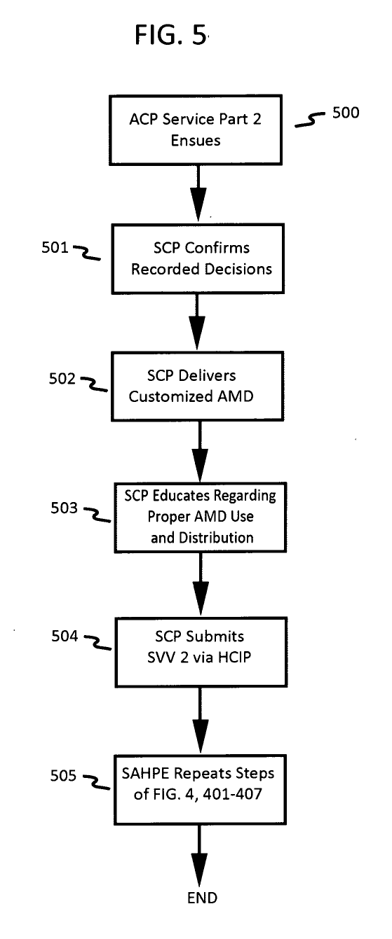

3.c. In accordance with FIG. 3, the SCP 102 completes and signs the Facilitator section of the Service Validation Voucher 301, and directs the client to rate the overall service, sign the form and initial the verification box to authenticate the time duration of the concluded education/conversation meeting. Then the SCP 102 works with the client to schedule the second, follow-up ACP conversation meeting 211, if any, in accordance with FIG. 5.

### Step 4: Documents Production and Transmission

4.a. In accordance with FIG. 3, the set of proprietary tools 201 are prepared for secure transmission to the SAHPE 101 by the SCP 102, using a computer-enabled internet network portal that is bilaterally encrypted for compliance standards established by HIPAA. In addition to the already completed SW of step 3.c. 301, which now is digitally scanned, the SCP crosswalks the conversation one script-prompted notes to the proprietary digital Case Notes Form 300, with both forms rendered in this first embodiment as locked fill-able Adobe Systems Portable Document Files and then transmitted 302.

4.b. In accordance with FIG. 3, upon receipt, the SAHPE 101 imports the embedded digital forms data from the submitted proprietary tools into a storage data base for digital and computational analysis and data manipulation 304 in order to facilitate compiled digital outputs to printer devices, other computing devices, and automated routing devices that will further transmit the analyzed digital file data securely and immediately to pre-determined and pre-authorized destinations and persons 305, including SAHPE-enrolled medical supervision practitioners whom the digital analysis process has screened against stored geographic proximity and pre-entered personal schedule availability data files for ACP consultations for which they are presently available via telephonic or computer interface, or as may be required by indicia from submitted SW data forms, a required face-to-face meeting with an individual client.

4.c. In accordance with FIG. 3, electronically or manually signed and scanned proprietary tool forms are transmitted by SAHPE-enrolled medical supervision practitioners 307 back to the SAHPE via the HIPAA secured portal 303, with confirmations, as necessary, of completed or scheduled face-to-face meetings and consultations with individual clients and/or their family members or surrogates indicated.

4.d. In accordance with FIG. 3, the SAHPE 101 receives all adjudicated and properly submitted data files 305, 306, 300 and creates from them the client's customized and QvQ-scored advance medical directive legal document in a form that is ready for printing and legal execution upon delivery 308. The SAHPE 101 then accesses the HIPAA-secure internet portal 303 to transmit the prepared document back to the SCP 102.

### Step 5: Insurance Claim Submitted

5.a. In accordance with FIG. 4, the SCP 102 receives the prepared customized and QvQ-scored advance medical directive back 400 via transmission through the HIPAA-secured internet portal 303. Thereupon the SAHPE 101 prepares and submits a Medicare insurance claim electronically to the appropriate Medicare Administrative Contractor entity 401 for completed ACP conversation one.

5.b. If the submitted claim is approved 402, the SAHPE 101, having prior agreed to accept benefits assignment and the Medicare allowable payment rate, the SAHPE 101 accepts the electronic banking deposit, but per Medicare regulations, also prepares a client invoice for any outstanding and collectible co-insurance or out-of-pocket deductible payments disclosed in the Medicare Explanation of Benefits Remittance Advice form, and the process ends 407.

5.c. In accordance with FIG. 4, if the submitted claim is denied 403 for disputable reasons, the claim is duly appealed 404 for reconsideration or re-opening.

5.d. If, in accordance with FIG. 4, the appealed claim is approved 405, then the description of 5.b. ensues and the process ends.

5.e. If, in accordance with FIG. 4, the appealed claim is denied 406, the process ends.

### Step 6: Conduct of Conversation Meeting 2

6.a. In accordance with FIG. 5, the SCP 102 convenes second client meeting 500.

6.b. In accordance with FIG. 5, the final document is reviewed by the client to ensure that all decisions, choices, and preferences were accurately captured, recorded, and reflected in the customized legal document 501.

6.c. Upon client review and approval, in accordance with FIG. 5, the finished legal document is handed over to the client 502, with instructional sheet inserts to its proper execution, distribution and use, as well as an enclosed information card cut-out for completion, copying, and lamination to keep on his or her person, in the glove compartment of the vehicle(s), and affixed by magnet to the home refrigerator 503. (In most instances, the SCP 102 is a public notary who will instruct the client to have two qualified witnesses meet them in order to legally execute the document at the present time. Absent this, the document is delivered from the SAHPE 101 with blanks for the client to enter he time when execution is independently arranged)

6.d. In accordance with FIG. 5, the SCP 102 and client complete the conversation two SVV form, which is then transmitted via HIPAA-secured internet portal 303 to the SAHPE 101 for claim submission two.

6.e. The SAHPE 101 next proceeds in accordance with FIG. 4, 401 through 407.

Although the present invention has been disclosed and described in detail for purposes of illustration, it is understood that such detail is solely for that purpose, and variations can be made therein by those skilled in the art without departing from the scope of the invention as expressed by the principles, descriptions, and drawings pertaining thereto. The present invention is described by the following claims.

What is claimed is:

1. A business method system and related means for making access to a Medicare-insured advance care planning education and conversation benefit more readily and widely available, on-demand, by means of a created alternate access pathway for Part B-covered beneficiaries to obtain the qualified benefits, from wherever and whenever they may independently choose, independent of any existing patient-doctor relationship, medical practice, hospital, or other medical-type facility or medical delivery location comprising:
a means for mobile delivery of comprehensive advance care planning, medical education, psychological/emotional support, and conversation services to the home-bound and non-ambulatory patient populations.
a means for mobile delivery of comprehensive advance care planning, medical education, psychological/emotional support, and conversation services to well and non-sick individuals, caregivers, family members, and decision making legal surrogates in homes, churches, other faith institutions, community centers, public libraries, residential communities for the aged, public restaurants, work sites, and a plurality of other non-medical systems locations where a personal doctor-patient relationship or the need for hospitalization does not exist.
a means for delivery of comprehensive advance care planning, medical education, psychological/emotional support, and conversation services by professionally trained and certified Advance Care Planning Facilitators.

2. A benefit delivery system in accordance with claim 1 that meets regulatory and compliance criteria for successful claims processing and insured benefits service reimbursements from the U.S. Centers for Medicare and Medicaid Services and from a plurality of private/commercial insurance carriers.
a means for clinical supervision of delivered advance care planning conversation benefits by actively licensed medical practitioners.
a means for medical documentation of a beneficiary/family member ACP encounter.
a means for time recording the duration of a beneficiary/family member ACP encounter.
a means for evaluating and rating the quality of a beneficiary/family member ACP encounter.

3. A set of specialized ACP tools in accordance with claim 2 comprising:
a means for recording clinical supervision, oversight and authorization of a beneficiary/family member ACP encounter.
a means for time-recording the duration of a beneficiary/family member ACP encounter.
a means for recording medical documentation of a beneficiary/family member ACP encounter.
a means for subjectively evaluating and rating the service quality of a beneficiary/family member ACP encounter.
a means for a certified professional ACP facilitator to record actions, participation and follow-up plans for a beneficiary/family member ACP encounter.
a means for recording, in advance, a quality-versus-quality-of-life preferences range for the directed termination of artificial life supports for a beneficiary's potential incapacity.
a means for recording, in advance, a predetermined period of trial on artificial life supports, absent any reasonable or acceptable medical recovery, and after which there shall be a termination of all such artificial life supports.
a means for recording signed understanding on the part of a beneficiary of regulatory disclosures and disclaimers prior to the initiation of a beneficiary/family member ACP encounter.

4. A business method system and related means in accordance with claim 1 for that uses a standardized ACP conversation script (not shown) and correlated case notes form (not shown) to create an individually-customized advance medical directive legal document resulting directly from the prior claimed method and system, further comprising:
a means for attempted protection of clients/patients and their family members, in advance, from the potential use of statutory Limitations on Actions that would bar a legal remedy or judicial recovery in the event of a willful and/or illegal disregard of a beneficiary's, his or her appointed Durable Medical Power of Attorney's, and/or his or her family members' statutory legal rights in an advance medical directive via a affidavit, called a “Time Bar Waiver Affidavit.”
a means for attempted protection of clients/patients and their family members, in advance, from the undisclosed and unauthorized extraction, use, and commercialization of their personal bodily fluids or tissues by other parties without advance permission from the client/patient's appointed Durable Medical Power of Attorney, called the “Henrietta Lacks Clause.”
a means for providing psychological/emotional comfort and support for clients/patients, family members, and others participating in difficult end-of-life conversations and medical realities, called “Happy Thoughts.”
a means for determining and recording, in advance, a client/patient's decision regarding an optional trial period for the use of artificial life supports or whether such period is to be decided in-the-moment by the client/patient's appointed Durable Medical Power of Attorney.
a means for determining and recording a client/patient's preferred hospital of choice in the event of a potentially prolonged hospital stay.
a means for determining and recording a client/patient's known medication and/or food allergies.
a means for determining and recording a client/patient's religious values, faith traditions, world view, and preferences for values-consistent final counseling, rituals, last rites, and prayer.
a means for determining and recording a client/patient's preference regarding the anatomical gift election.
a means for determining and recording a client/patient's preference regarding final disposition burial versus cremation.
